(19) United States
(12) Patent Application Publication
(10) Pub. No.: US 2007/0225773 A1
(43) Pub. Date: Sep. 27, 2007

(54) IMPLANTABLE TRANSCRANIAL PULSE GENERATOR HAVING A COLLAPSIBLE PORTION

(76) Inventors: Yang Shen, London (CA); Souhile Assaf, London (CA); Mandar Jog, London (CA)

Correspondence Address: BRUNET & CO. LTD., 10712 MELROSE DR., KOMOKA, ON, N0L-1R0, CA

(21) Appl. No.: 11/536,359

(22) Filed: Sep. 28, 2006

Related U.S. Application Data
(60) Provisional application No. 60/596,501, filed on Sep. 28, 2005.

(51) Int. Cl. A61N 1/00

(52) U.S. Cl. 607/45

(57) ABSTRACT

An implantable transcranial pulse generator for generating neuro-modulating electrical signals used, for example, in the treatment of medical conditions through deep brain stimulation (DBS). The implantable pulse generator comprises a collapsible dome portion that deforms upon impact to protect the patient from injury and the pulse generator from being damaged. The dome is removably mounted to a transcranial insert that is secured within a burr hole located in the patient's cranium. Both the dome and the insert contain electronic components and have complementary connectors facilitating direct electrical interconnection. The electronics within the dome are mounted on flexible substrates to permit deformation of the collapsible portion. The dome may include a re-fillable reservoir for supplying controlled dosages of a pharmaceutically active composition to the brain through the transcranial insert.

### CROSS-REFERENCE TO RELATED APPLICATIONS

This application claims the benefit of U.S. patent application No. 60/596,501, filed Sep. 28, 2005, which is incorporated herein by reference.

### FIELD OF THE INVENTION

The invention relates to implantable transcranial pulse generators for the generation of neuro-modulating electrical signals used, for example, in the treatment of medical conditions through deep brain stimulation (DBS). More particularly, the present invention relates to an implantable pulse generator having a collapsible dome portion that deforms upon impact to protect the patient from injury and to protect the pulse generator from being damaged.

### BACKGROUND

The DBS technique is gaining acceptance as a method of treating medical conditions such as chronic pain, Parkinson's disease, migraine headache and depression. In the DBS technique, an electrode is implanted within the brain and an electrical pulse is applied therethrough in order to modulate neurological activity in the vicinity of the electrode. One or more electrodes may be used in any given therapeutic setting and these electrodes may be connected to a common pulse generator or separate pulse generators. The pulse generator(s) may be worn externally or may be implanted in the body of the patient. Implanted pulse generators may be located either externally to the cranium, within the cranium, or a combination of the two referred to as a transcranial pulse generator. The various types of neurological pulse generators have advantages and disadvantages depending on their intended use and are not necessarily applicable in all situations. For transcranial pulse generators, it is important that they be small in size and have relatively little in the way of emitted electromagnetic interference (EMI), since this can cause havoc with the proper implementation of the DBS technique. A comprehensive discussion of the DBS technique commenting on different types of pulse generators can be found in Bittar, R. G., Burn, S. C., et al., “Deep brain stimulation for movement disorders and pain”, Journal of Clinical Neuroscience, vol. 12, no. 4, pp. 457-463, 2005, and in Chang, J.-Y., “Brain stimulation for neurological and psychiatric disorders, current status and future direction”, Journal of Pharmacology and Experimental Therapeutics, vol. 309, pp. 1-7, 2004, which are incorporated herein by reference.

For implantable transcranial pulse generators, cranial trauma can result in damage to the pulse generator and/or injury to the patient. Dislodgment of the pulse generator or the implanted electrodes due to impact can cause tissue damage, a head wound, or can simply render future DBS treatment ineffective. It would therefore be desirable to provide an impact resistant implantable pulse generator in order to mitigate some or all of these effects.

In addition, should an implanted pulse generator become damaged, it would be desirable to remove or replace the damaged portion without having to perform extensive surgery.

As an adjunct to the DBS technique, it is often necessary to administer certain pharmaceutical compositions to the brain tissue in the vicinity of the electrode. This is often difficult to do in practice, since it is difficult to administer the drugs transcranially to the desired location and at the desired dosage level and frequency. It would therefore be desirable to provide a method of transcranial drug delivery that administers controlled dosages of drugs to the vicinity of the electrodes. It would further be desirable to utilize existing hardware implanted for implementing the DBS technique.

United States patent publication 2005/0143790 discloses an intracranial neural interface system. The interface comprises a chamber having a cranial insert piece and a top piece that are electrically connected to one another by means of a cable. The interface also includes means for chemical delivery. Both the cranial insert piece and the top piece are rigid, with sharp edges that can irritate the scalp as it moves over the interface. The pieces are provided as a single connected unit, rather than two separate pieces, so the surgeon must be careful not to damage the physical connection during installation. Should this physical connection become damaged due to impact after installation, the insert piece and top piece could separate from one another. All of the electronics are provided outside of the cranium within rigid housings; any impact to the interface would cause it to shatter, allowing the electronics to migrate freely under the scalp. Furthermore, a ribbon cable passing through a lumen in the casing is used to connect the extracranial electronics to the intracranial electrode. Since there is no intracranial connector on the insert for attachment to the electrodes, the number of electrodes that can be connected is limited to the size of the burr hole opening, which is usually kept as small as possible. In addition, impact to the head could cause the electrodes to be dislodged, since they are not secured to any part of the interface. This system is therefore not impact resistant and provides opportunity for injury should impact occur.

United States patent publication 2005/0075680 discloses methods and systems for intracranial neurostimulation and/or sensing. The device used is essentially a screw passing through the skull that is used to conduct stimulation pulses. The conductive screw still must be connected to an electrical pulse generator in order to function as a neurostimulator, and this extracranial pulse generator is susceptible to impact damage and/or connection loss. In addition, the portion located beneath the scalp has a large protrusion with a sharp edge, which can cause scalp irritation or cause the scalp to become cut upon impact.

U.S. Pat. No. 6,553,263 discloses implantable pulse generators using rechargeable zero-volt technology lithium-ion batteries. The batteries used in this design have a rigid casing that encloses fluidic electrolytes; the batteries are therefore susceptible to leakage upon impact. Leakage of the battery materials into the cranium could be fatal, therefore this design is not particularly impact friendly.

United States patent publication 2004/0121528 discloses an electronic unit integrated into a flexible polymer body. Although this device discloses some interesting concepts that could lead to non-rigid polymeric electronics substrates, impact resistance is not discussed and this invention is not directed to the field of implantable transcranial pulse generators.

U.S. Pat. No. 5,833,709 discloses a method of treating movement disorders by brain stimulation. Although this is not directed to implantable transcranial pulse generators, DBS techniques are discussed in general.

Heretofore, none of the available implantable transcranial pulse generators have addressed the problems in the art related to impact resistance, replaceability and pharmaceutical delivery. A need therefore still exists for an improved implantable transcranial pulse generator that addresses some or all of the problems of the prior art.

### SUMMARY OF THE INVENTION

According to the present invention, there is provided an implantable electrical pulse generator for neurological stimulation of a brain of a patient comprising a dome for mounting beneath a scalp of the patient, the dome comprising at least a collapsible portion. The implantable pulse generator may further comprise a transcranial insert for mounting within a burr hole located in a skull of the patient. The transcranial insert may be integrally formed with the dome and downwardly depend from an underside thereof or may be separable from the dome. This permits the transcranial insert to be secured to the patient's skull and the dome to be removably mounted thereto above the skull and beneath the scalp. This permits the dome to be replaced in the event that it becomes damaged using a minimally invasive surgical procedure.

The collapsible portion may be flexible and may be made from, for example, an elastomeric material. The collapsible portion may be fluid or gel filled or may comprise a hollow interior. The collapsible portion may be resiliently biased away from the cranium (i.e. towards the scalp). The invention may include a flexible interior diaphragm that may be resiliently biased towards the collapsible portion so that, upon impact, both the collapsible portion and the diaphragm may be deformed away from the impact, for example towards the skull. The diaphragm may deform into an unoccupied area of the dome or the insert. Following impact, the diaphragm may then resiliently return to its original shape and thereby urge the collapsible portion away from the skull. The dome may be secured to the insert by a friction-fit connection, rather than a mechanical connector or fastener, in order to advantageously reduces the likelihood of damage to the connection and the possibility of the dome becoming loose. The insert may be rigid or semi-rigid and may be secured to the skull using suitable fasteners. Both the dome and the insert may be made from materials that are substantially non-interfering with magnetic resonance imaging (MRI) techniques.

The dome, the insert, or both may contain electronic components. For example, the dome may contain a microprocessor, may contain electronic components suitable for generating a neuro-modulating electrical pulse and/or may contain electronic components suitable for recording neurological signals generated by brain tissue. Some or all of the electronics within the dome may be mounted on a flexible interior substrate. The flexible interior substrate may comprise a flexible film battery. The mounting of electronics on flexible substrates allows movement upon deformation of the collapsible portion, thereby preventing the electronics from becoming damaged. The dome may comprise a helical coil as part of the collapsible portion and the helical coil may be made from a flexible substrate. The dome may comprise an electrical connector for direct interconnection with a complementary connector on the insert. The insert may comprise an intracranial connector for connection to one or more electrodes implanted within the brain.

The dome may comprise a fluidic connection to the transcranial insert that may be used, for example, to supply a pharmaceutically active composition to the brain. The dome may comprise a reservoir for containing the pharmaceutically active composition. The reservoir may contain a liquid or a gel and may comprise a hollow compartment or a sponge. The reservoir may be refillable using, for example, a hypodermic needle. The dome may be self-sealing upon withdrawal of the hypodermic needle to prevent leakage of the pharmaceutically active composition from the reservoir. The implantable pulse generator may further comprise metering means for administering the pharmaceutically active composition at a controlled dosage. The metering means may comprise a pump means, a valve means, a means for squeezing or otherwise pressurizing the reservoir, a peristaltic fluid transfer mechanism, or any other suitable system. The pharmaceutically active composition may be administered to the epidural space, the cortex, the scalp, or the brain. For example, the composition may be administered to the brain directly in the vicinity of one or more electrodes.

### BRIEF DESCRIPTION OF THE DRAWINGS

Having summarized the invention, preferred embodiments thereof will now be described with reference to the accompanying figures, in which:

FIG. 1 shows an implantable pulse generator according to the present invention mounted transcranially in a patient and connected to a single DBS electrode;

FIG. 2 shows a cross-section of an implantable pulse generator according to the present invention;

FIG. 3a shows the implantable pulse generator of FIG. 2 in a normal position;

FIG. 3b shows the implantable pulse generator of FIG. 2 in a deformed position;

FIG. 4a shows an exploded view of an implantable pulse generator according to the present invention illustrating one embodiment of an electrical connector for directly interconnecting the dome with the insert;

FIG. 4b shows an exploded view of an implantable pulse generator according to the present invention illustrating another embodiment of an electrical connector for directly interconnecting the dome with the insert;

FIG. 5 shows a helical flexible substrate for use with a dome for an implantable pulse generator according to the present invention comprising; and,

FIG. 6 shows an embodiment of a dome for an implantable pulse generator according to the present invention comprising a reservoir and metering means for administering a pharmaceutical composition.

### DETAILED DESCRIPTION

In the following description, like features of the drawings will be referred to using like reference numerals. Accordingly, not all features labeled on a particular drawing need necessarily be described with reference to that particular drawing, but will be described with reference to at least one of the drawings.

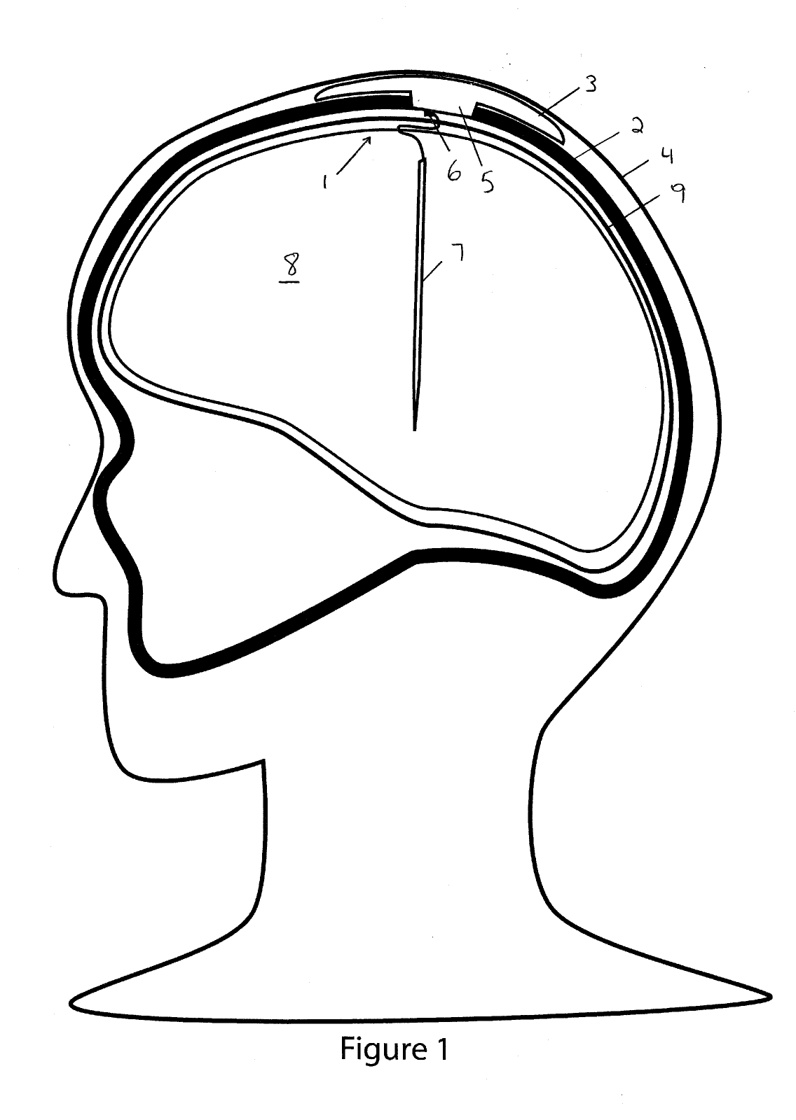

Referring to FIG. 1, a patient undergoing DBS treatment has an implantable pulse generator 1 installed transcranially through a burr hole in the skull 2. The implantable pulse generator 1 has a dome 3, located between the skull 2 and the scalp 4, and a transcranial insert 5. The transcranial insert 5 has an intracranial connector 6 at the bottom thereof for connection to an electrode 7 implanted within the tissue of the brain 8. The insert 5 may be connected to multiple electrodes 7, which may vary in size and location. Since the brain 8 is able to move within the skull 2, the electrode 7 is connected to the connector 6 by means of a small diameter cable. This desirably reduces the size of the opening in the dura 9 that is required to permit passage of the cable and thereby decreases the likelihood of cerebral spinal fluid (CSF) leakage. The dome 3 is relatively flat, but much larger in diameter than the insert 5. This reduces bulging of the scalp 4 and spreads out the collapsible area of the dome 3, making it more readily able to absorb impact.

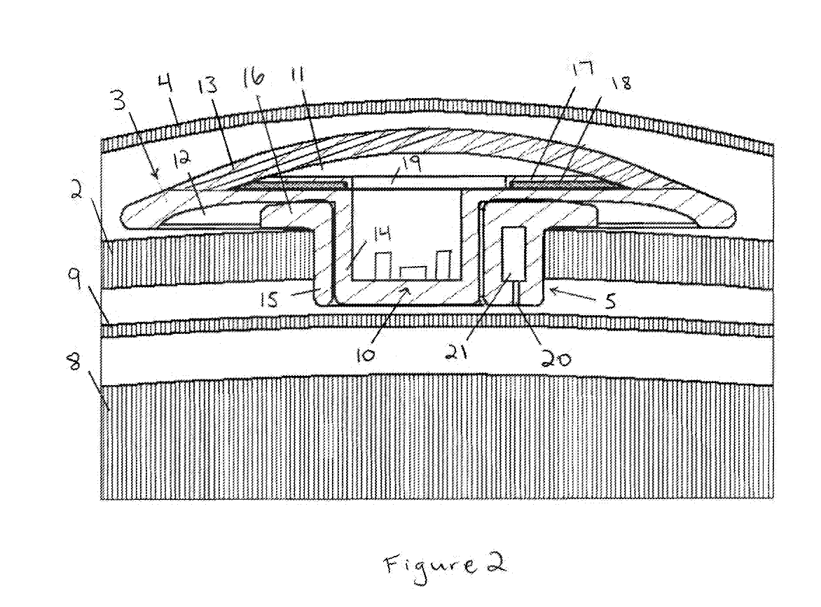

Turning to FIG. 2, the dome 3 is located beneath the scalp 4 and above the skull 2. The dome 3 includes a downwardly depending plug 14 for insertion within a complementary receptacle 15 of the transcranial insert 5. The interior of the plug 14 contains electronics 10 suitable for generating a neuro-modulating electrical pulse or for recording neurological activity in the brain. The insert 5 also includes a concentric ring 16 which abuts the exterior surface of the skull and prevents the insert 5 from passing through the burr hole. When the plug 14 is inserted into the complementary receptacle 15 to install the dome 3, the ring 16 resides within a concavity 12 located on the underside of the dome 3.

Although in this embodiment the entire dome 3 is flexible, the main collapsible portion 13 is located in approximately the centre of the dome 3 above a hollow chamber 11. Upon deformation of the centre collapsible portion 13, the circumferential edges of the dome 3 have a tendency to rise relative to the skull 2. In addition to accommodating the ring 16, the concavity 12 also permits this upward edge movement to happen more readily.

The hollow chamber 11 houses electronic components 17 mounted on a flexible substrate 18. In one embodiment, the flexible substrate comprises a flexible film battery that provides excellent energy storage and rechargeability while not being susceptible to fluid leakage or impact damage. Also located within the hollow chamber 11 is a flexible diaphragm 19. The flexible diaphragm 19 normally resides in a neutral or planar position, but may alternatively be resiliently biased upwardly towards the collapsible portion 13. The diaphragm 19 may be a separate component or may be integrally formed with the dome 3. The diaphragm 19 may be made from a semi-rigid material or an elastomeric material. The hollow cavity 11 may be filled with an electrically non-conductive fluid, preferably a non-leaking fluid such as a gel with a high dielectric constant. Any fluid used in the implantable pulse generator is preferably bio-compatible to reduce the risk of adverse patient consequences in the event of leakage.

The insert 5 may include electronic components located on the ring 16 or at any other suitable location. The insert 5 includes an intracranial connector 20 located on an underside thereof for effecting an electrode connection in the epidural space between the dura 9 and the skull 2. Although the electrode and connecting cable have been omitted for clarity, the cable would normally extend through the dura 9 and the electrode would normally be located in the brain tissue 8 as previously described with reference to FIG. 1. An insert cavity 21 may contain switches or other circuitry necessary to effect connection to one or more electrodes via the connector 20. The cavity also includes a portion of the electrical connection means used to directly interconnect the dome 3 and the insert 5, as will be more thoroughly described hereinafter.

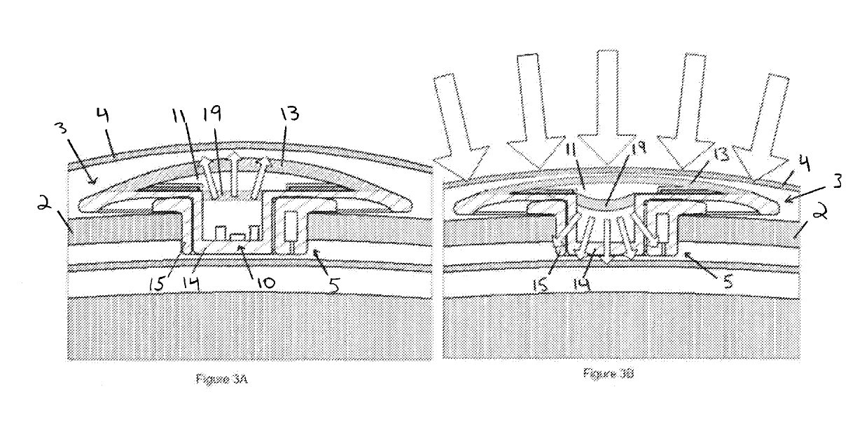

Referring to FIG. 3a, in its normal position the diaphragm 19 is planar and exerts only a minor upward bias against the collapsible portion 13 through fluid pressure in the cavity 11. Turning to FIG. 3b, upon impact deformation of the collapsible portion 13, the flexible diaphragm 19 is resiliently displaced downwardly into the area of the plug 14 that is unoccupied by pulse generating electronics 10 due to an increase in fluid pressure in the hollow cavity 11. This absorbs and dissipates the impact energy, reducing the likelihood of damage to the generator or the patient. Upon cessation of the impact, the resilience of the diaphragm 19 causes it to return to its original shape, thereby further increasing fluid pressure in the cavity 11 and urging the collapsible portion 13 away from the skull 2 and back toward its original shape. Use of an inert gel or similar non-compressible biocompatible fluid increases the efficacy of energy transfer in this system.

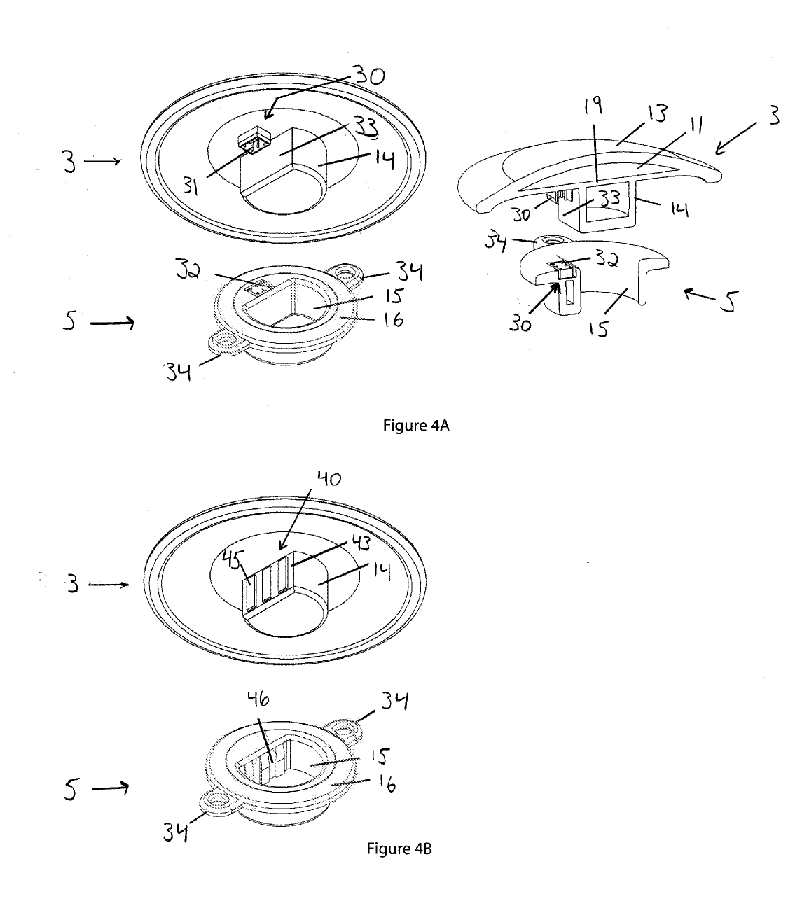

Referring to FIG. 4a, an exploded view of an implantable pulse generator is shown with an embodiment of an electrical connector 30 that permits direct interconnection between the insert 5 and the dome 3. In the embodiment shown, the connector 30 comprises a set of pins 31 extending from the underside of the dome 3 for lodgment within complementary apertures 32 in an upper surface of the ring portion 16. In order to ensure properly alignment of the pins 31 with the apertures 32, the plug 14 includes a chordal chamfer 33 that ensures it can only be inserted within the receptacle 15 in a single orientation. The ring 16 includes two securement tabs 34 that are used in fastening the insert 5 to the skull 2.

FIG. 4b shows an alternative embodiment of an electrical connector 40 that permits direct interconnection between the insert 5 and the dome 3. In this embodiment, the plug 14 includes a chordal chamfer 43 that ensures it can only be inserted within the receptacle 15 in a single orientation. The chordal chamfer 43 includes a set of raised conductive pads 45 that form a sliding connection within grooves 46 having complementary conductive recessed surfaces. Either the pads 45 or the grooves 46 may be resiliently biased towards one another to ensure intimate contact and electrical connection takes place. Persons skilled in the art will recognize that, in an alternative configuration, the plug 14 could comprise the grooves 46 with the receptacle 15 containing the pads 45. One advantage of the sliding interconnection afforded by this embodiment is that it is able to accommodate relative movement between the dome 3 and the insert 5 upon impact deformation without interrupting or damaging the electrical interconnection.

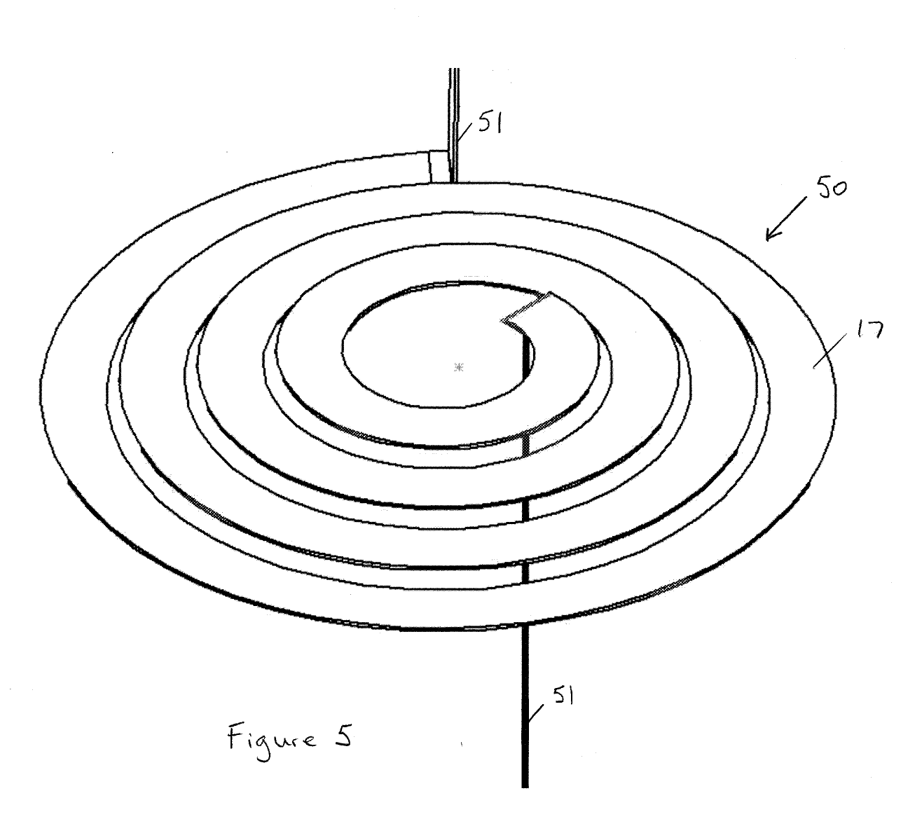

Referring to FIG. 5, an alternative embodiment of a dome 3 comprises a flexible helical coil 50 provided within the chamber 11. The helical coil 50 is intrinsically resiliently biased away from the skull and therefore obviates the need for a diaphragm 19. The helical coil 50 may be made from or made incorporating the flexible electronic substrate 17 and/or the flexible film battery 18. In this manner, the number of components within the cavity 11 is reduced, with overall space savings. The helical coil 50 may therefore comprise electrical connections 51 at its ends, preferably adapted for sliding interconnection with the insert or the remainder of the dome 3.

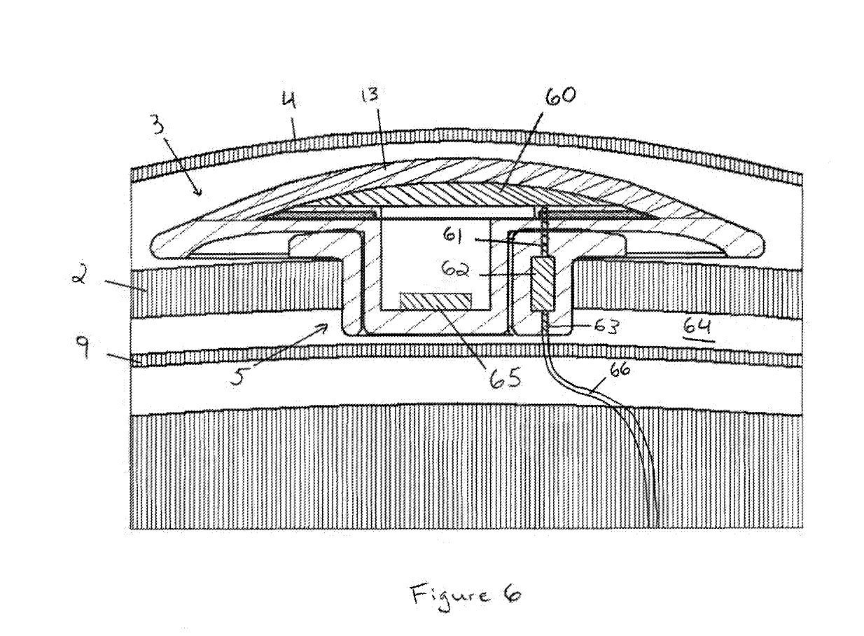

Referring to FIG. 6, an embodiment of the present invention is shown wherein a pharmaceutically active composition is provided within a reservoir 60 located within the cavity 11. The reservoir 60 is in fluid communication with the insert 5 through fluid conduit 61. A metering means 62, which in this embodiment is a pump, is provided along the conduit 61 to control the rate of delivery of the composition. The metering means 62 may be controlled by a microprocessor 65 located within the plug 14. The metering means 62 also provides a high resistance to flow to prevent an inadvertent overdose of the pharmaceutical composition upon impact deformation of the reservoir 60. In order to prevent inadvertent leakage, the reservoir 60 may comprise a sponge-like material. This has the additional benefit of reducing the effect of reservoir depletion on cavity volume, which could have negative consequences for impact absorption.

In order to refill the reservoir 60, a hypodermic needle may be inserted through the scalp 4 and through the collapsible portion 13 into the reservoir 60. Upon removal of the needle, the collapsible portion 13 self-heals in order to prevent leakage. This provides an effective and expedient means for replenishing the reservoir 60 without requiring the surgical removal of the dome 3. The microprocessor 65 may be used to program the metering means 62 to deliver the pharmaceutical composition using a pre-determined dosage profile. The pharmaceutical composition may be conveyed through the conduit 61 either directly into the epidural space 64 or into an epidural fluid connector 63 used to attach a catheter 66 for delivery into a desired location within the cranium. This catheter could be co-located with the electrode to target tissue in the vicinity of stimulation with minimal ancillary tissue trauma.

The foregoing describes preferred embodiments of the invention and other features and embodiments of the invention will be evident to persons skilled in the art. The following claims are to be construed broadly with reference to the foregoing and are intended by the inventor to include other variations and sub-combinations, even if not explicitly claimed.

1) An implantable electrical pulse generator for neurological stimulation of a brain of a patient comprising a dome for mounting beneath a scalp of the patient, the dome comprising at least a collapsible portion.

2) The implantable electrical pulse generator of claim 1, further comprising a transcranial insert for mounting within a burr hole located in a skull of the patient.

3) The implantable electrical pulse generator of claim 2, wherein the dome is separable from the transcranial insert and is mounted to the transcranial insert above the skull and beneath the scalp of the patient.

4) The implantable pulse generator of claim 1, wherein the dome further comprises a flexible interior diaphragm.

5) The implantable pulse generator of claim 4, wherein the diaphragm is resiliently biased towards the collapsible portion.

6) The implantable pulse generator of claim 5, wherein, upon impact, both the collapsible portion and the diaphragm are deformed and, following impact, the diaphragm resiliently returns to its original shape and thereby urges the collapsible portion away from the skull.

7) The implantable pulse generator of claim 4, wherein the diaphragm is deformable into an unoccupied area of the dome.

8) The implantable pulse generator of claim 3, wherein the dome comprises an electrical connector for direct interconnection with a complementary connector on the transcranial insert.

9) The implantable pulse generator of claim 1, wherein the dome contains at least one electronic component mounted on a flexible interior substrate.

10) The implantable pulse generator of claim 9, wherein the flexible interior substrate comprises a flexible film battery.

11) The implantable pulse generator of claim 1, further comprising electronic components suitable for generating a neuro-modulating electrical pulse.

12) The implantable pulse generator of claim 1, further comprising a microprocessor.

13) The implantable pulse generator of claim 2, wherein the transcranial insert comprises an intracranial connector for connection to an electrode implanted within the brain.

14) The implantable pulse generator of claim 2, wherein the dome comprises a reservoir containing a pharmaceutically active composition.

15) The implantable pulse generator of claim 14, wherein the reservoir is refillable by hypodermic needle injection and is self-sealing upon withdrawal of the hypodermic needle.

16) The implantable pulse generator of claim 14, further comprising metering means for administering the pharmaceutically active composition at a controlled dosage.

17) The implantable pulse generator of claim 14, wherein the dome comprises a fluidic connection to the transcranial insert.

18) The implantable pulse generator of claim 17, wherein the transcranial insert comprises a catheter connector.

19) The implantable pulse generator of claim 1, wherein the collapsible portion is resiliently biased toward the scalp.

20) The implantable pulse generator of claim 1, wherein the dome comprises a helical coil.
